(19) United States
(12) Patent Application Publication
(10) Pub. No.: US 2018/0008184 A1
(43) Pub. Date: Jan. 11, 2018

(54) METHOD TO EXERCISE EYES, HANDS AND FEET COORDINATION

(71) Applicants: James Winter Cole, Richardson, TX (US); Alfred M. Saleh, Richardson, TX (US)

(72) Inventors: James Winter Cole, Richardson, TX (US); Alfred M. Saleh, Richardson, TX (US)

(21) Appl. No.: 15/711,245

(22) Filed: Sep. 21, 2017

(51) Int. Cl. A61B 5/16; A61H 5/00; G09B 7/00; A61B 3/028; A63B 69/00

(52) U.S. Cl. CPC A61B 5/162; G09B 7/00; A61B 3/028; A61H 5/00; A63B 2069/0055

(57) ABSTRACT

Currently there is no apparatus or method to exercise mental agility by the simultaneous and/or independent control from the hands, feet, and eyes.

As depicted in FIGS. 1-7, the eye detects that the foot is slower than the hand and the information is sent immediately from the eye to the brain. Subsequently, the brain will instantaneously instruct the foot to speed up and stay on track in the template. The method can be repeated to ensure that the cursors have reached the target at the same time. Repetition enhances the coordination and reflection of the user.

### BACKGROUND OF THE INVENTION

Currently there is no apparatus or method to exercise mental agility by the simultaneous and/or independent control from the hands, feet, and eyes.

### BRIEF SUMMARY OF THE INVENTION

A method to enhance mental agility by exercising the simultaneous and/or independent control from the actions each hand and foot.

### BRIEF DESCRIPTION OF THE DRAWINGS

FIG. 1. FIG. 1—Simultaneously move the cursors from each corner of the screen to the middle square by using your left hand, right hand, left foot and right foot

FIG. 2. Simultaneously move the cursors in each square in the same direction of the arrows. Start from A to B to C to D to A.

FIG. 3. Simultaneously move the cursors in each square in the same direction of the arrows. Start from D to C to B to A to D.

FIG. 4. Simultaneously move the cursors in each triangle in the same direction of the arrows. Start from A to B to C to A.

FIG. 5. Simultaneously move the cursors in each triangle in the same direction of the arrows. Start from C to B to A to C.

FIG. 6. Simultaneously move the cursors clockwise in each star in the same direction of the arrows.

FIG. 7. Simultaneously move the cursors counter-clockwise in each star in the same direction of the arrows.

### DETAILED DESCRIPTION AND BEST MODE OF IMPLEMENTATION

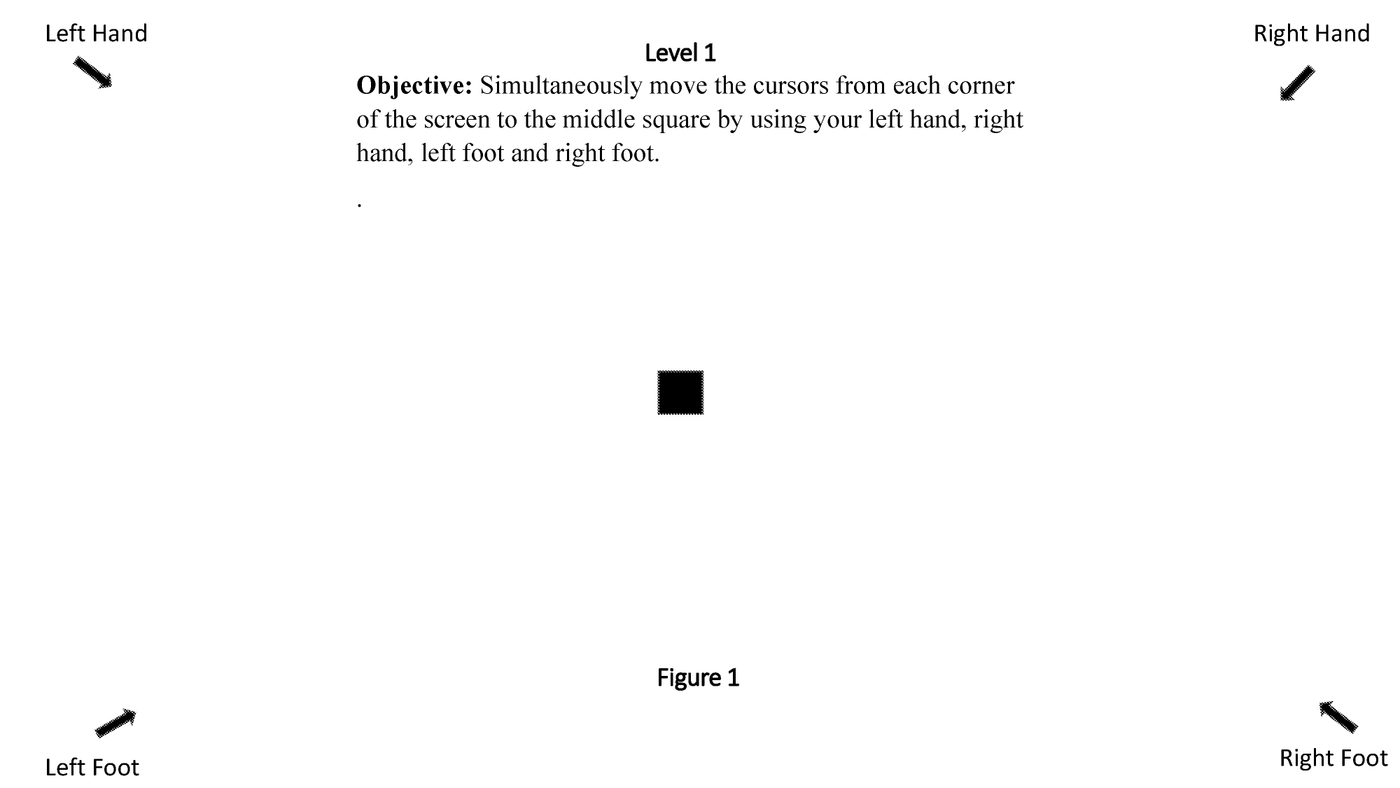

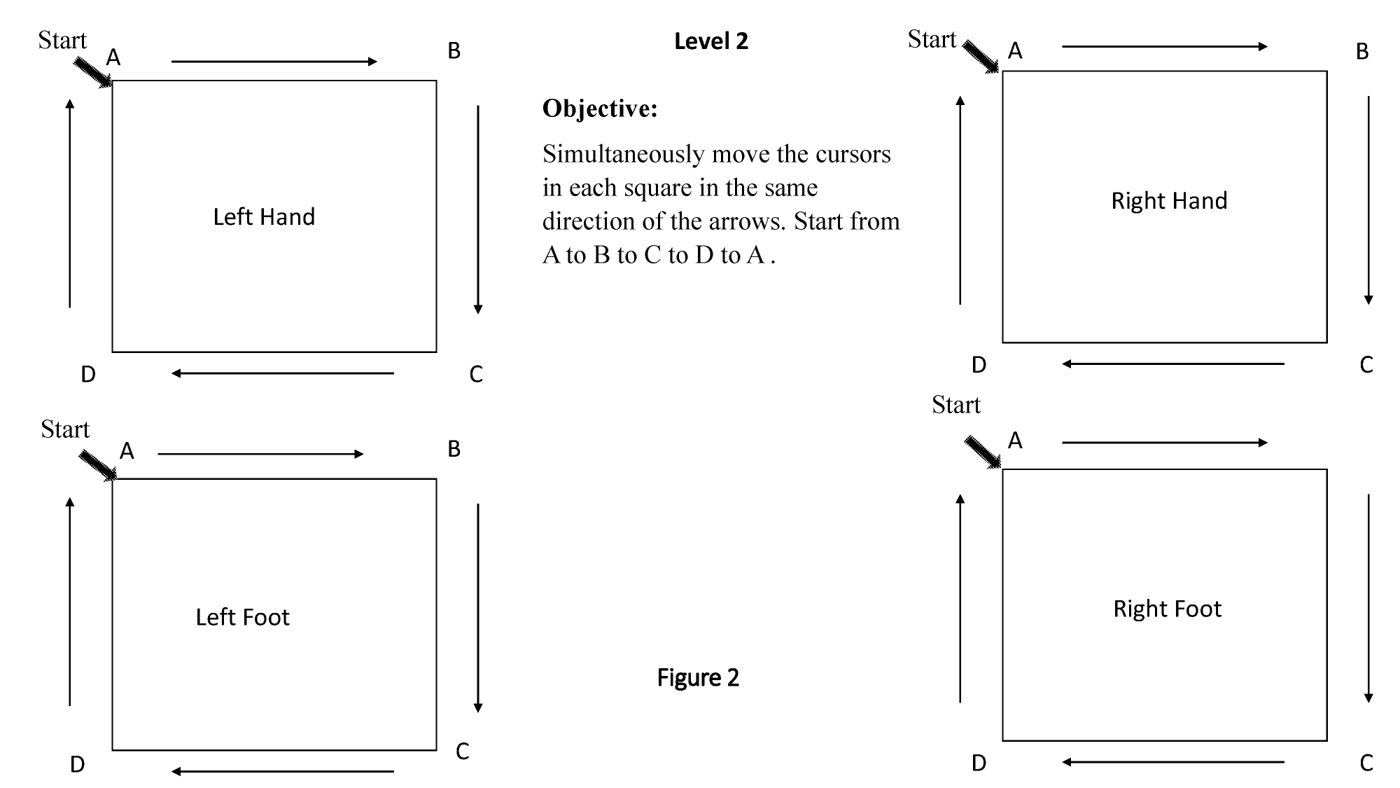

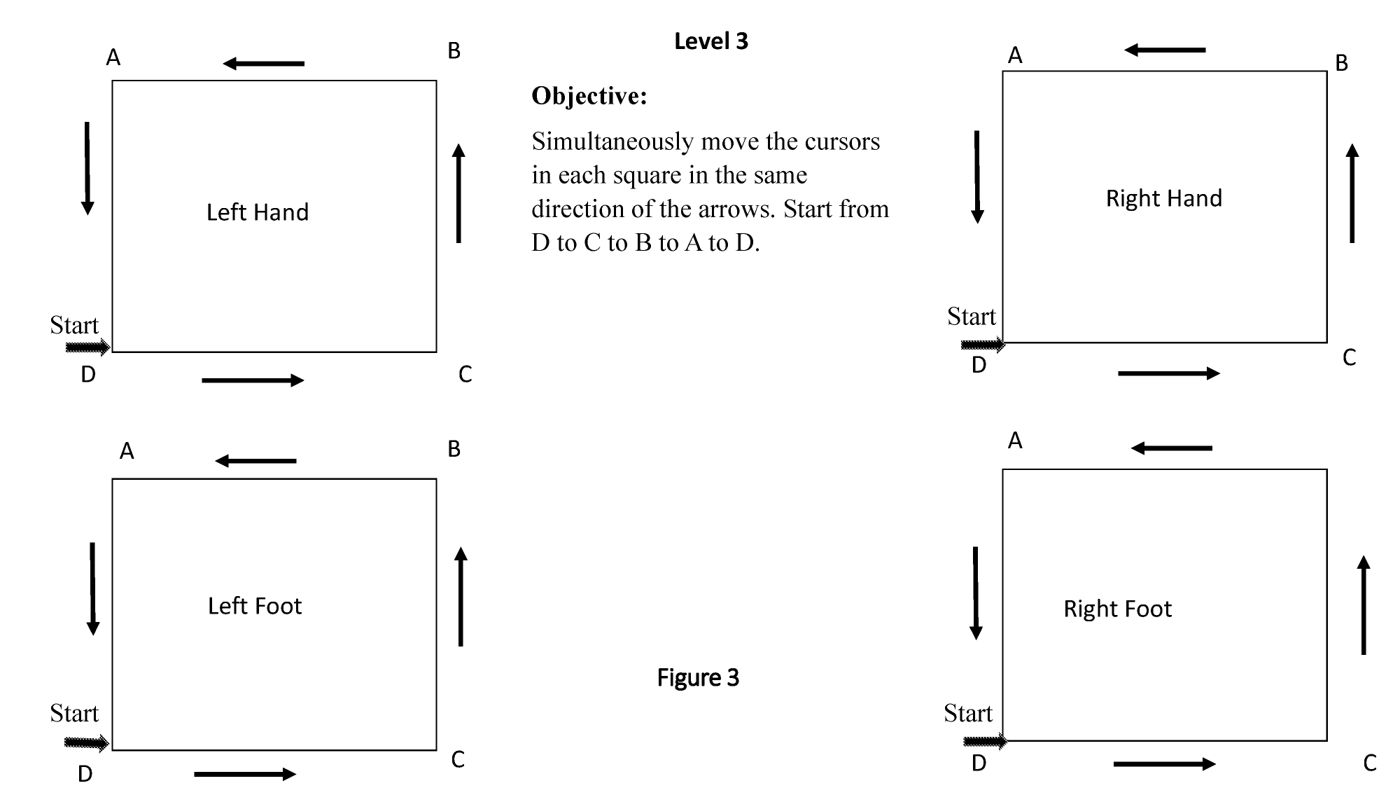

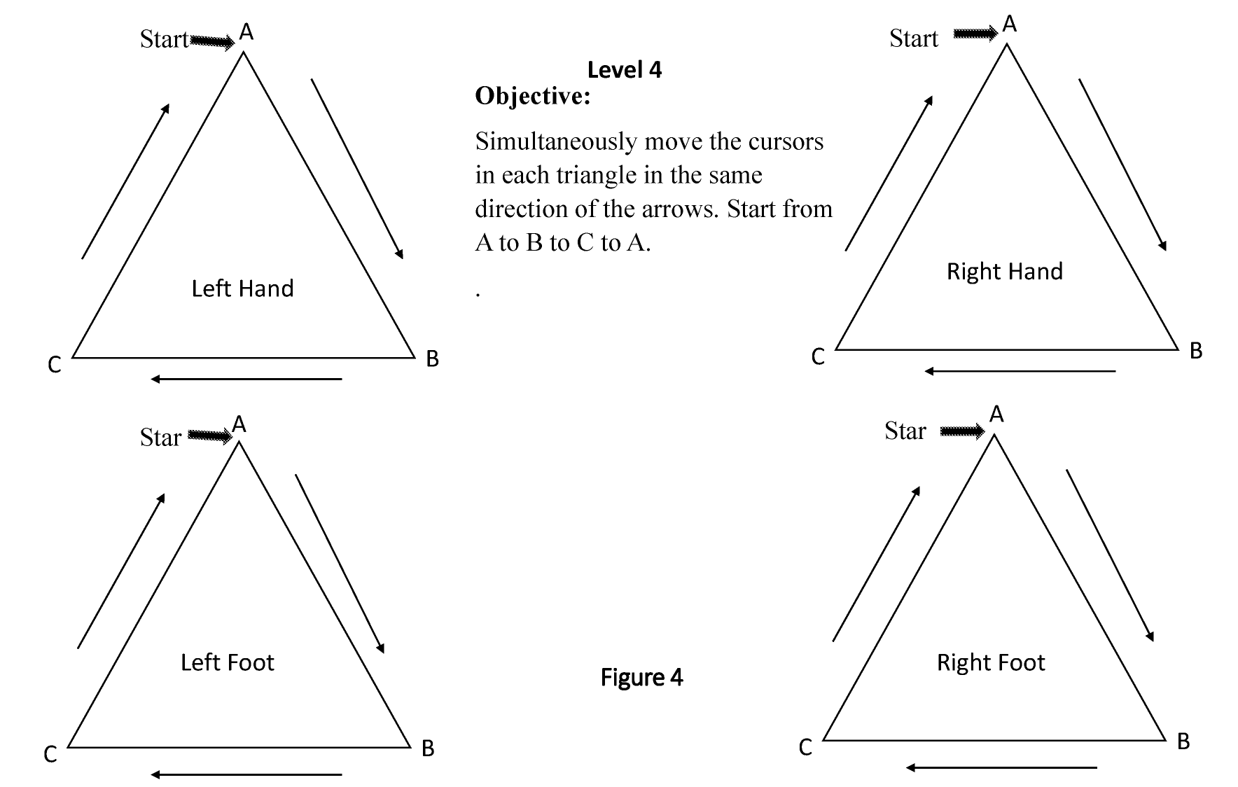

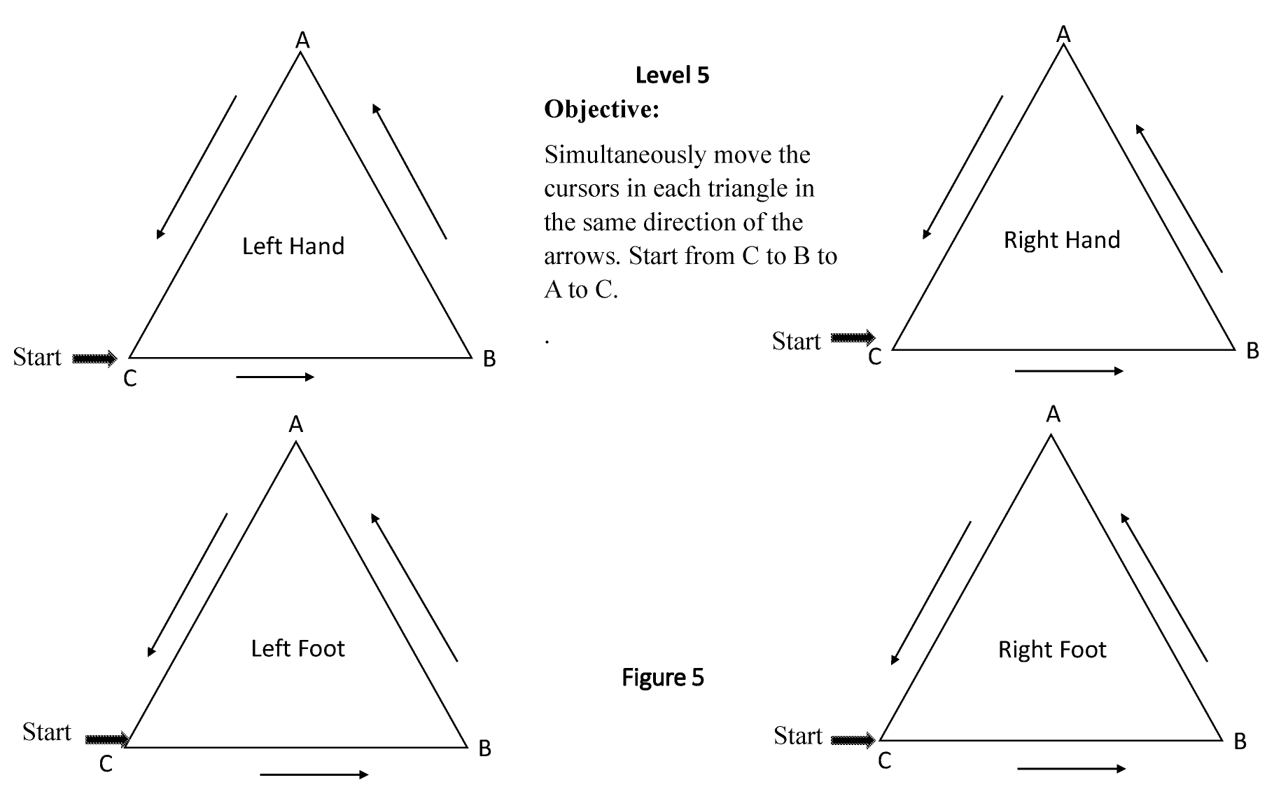

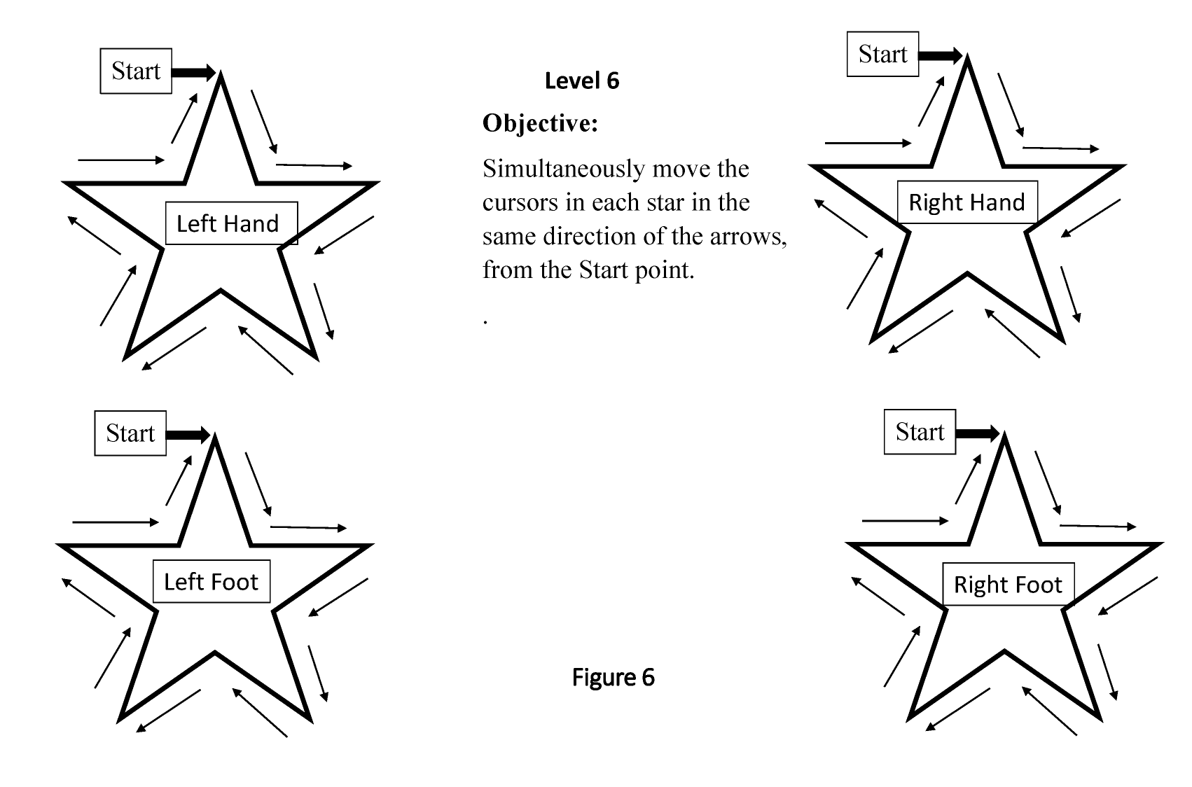

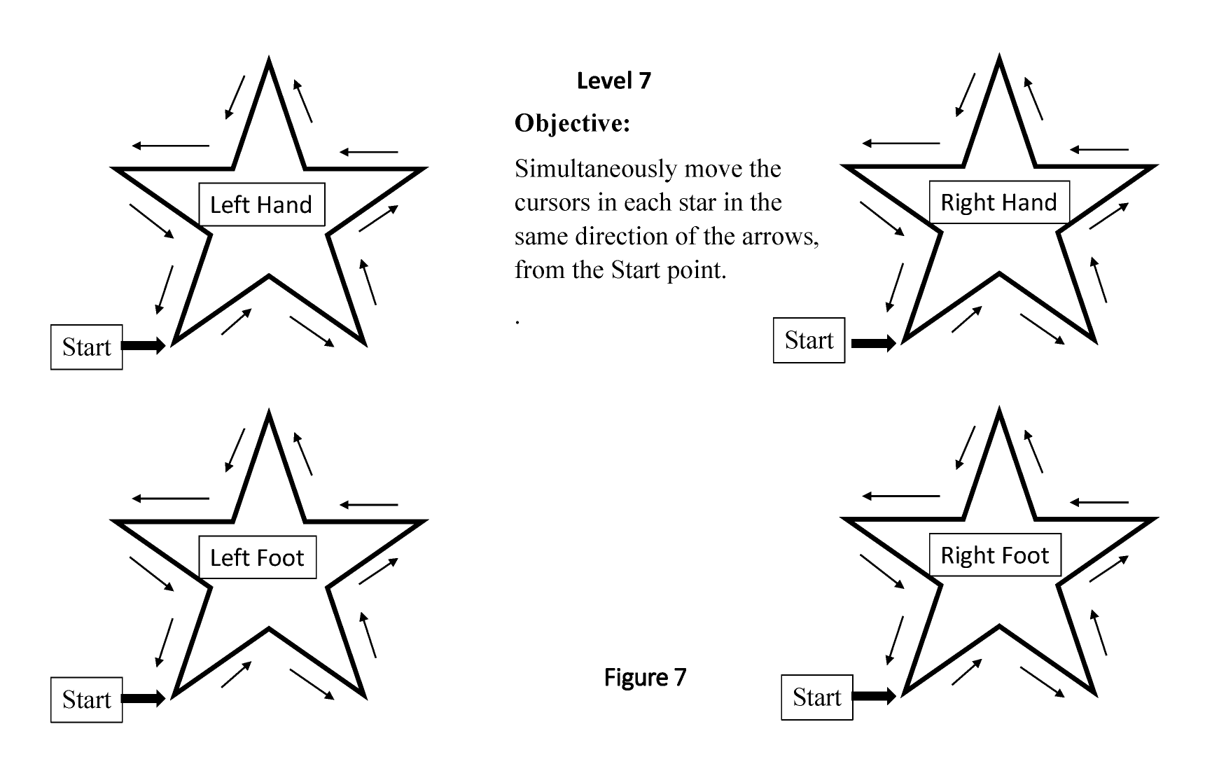

A new method to exercise mental agility by assigning and labeling a computer cursor to the left hand, right hand, left foot and right foot. Each computer cursor will be independently and/or simultaneously controlled by a wire or wireless hand and foot computer mice. The objective is to reach a designated target from a defined starting point by tracing the templates displayed on the screen using the cursors as depicted in FIGS. 1-7. All templates are designed to a specific challenge. Measuring the speed of each cursor will be displayed, recorded, and stored for retrieval by the user to determine and compare the responsiveness of eye coordination against the hands and feet. Repetition will reduce target access time for each cursor to reach the target at the same time. Cursor data information, such as speed, will be displayed on the screen to measure progress and challenges. The method will include two (2) hand mice and two (2) foot mice, and is not limited to operating systems such as Windows, Apple iPod, Blue Ray players, tablets, smart phones, smart TVs, or other simulator devices.

1. We claim a method using computerized cursors controlled simultaneously and/or independently by the hands and feet: This method provides the information related to the speed of each cursor that exercises user mental agility to accomplish a given result. The eye compares the slower cursor against the faster cursor and in turn the brain instructs the slower cursor to match the speed of the faster cursor to achieve the result as depicted in FIGS. 1-7. The hands and feet mice perform this method of cursor control. Repeating the exercise improves user performance, coordination, and reflection.
